(19) United States
(12) Patent Application Publication
(10) Pub. No.: US 2012/0107389 A1
(43) Pub. Date: May 3, 2012

(54) AMPHOTERIC LIPOSOMAL COMPOSITIONS FOR CELLULAR DELIVERY OF SMALL RNA MOLECULES FOR USE IN RNA INTERFERENCE

(76) Inventors: Jayanta Bhattacharyya, Hyderabad (IN); Arabinda Chaudhuri, Hyderabad (IN)

(21) Appl. No.: 13/262,013

(22) PCT Filed: Mar. 19, 2010
(86) PCT No.: PCT/IN2010/000164

§ 371 Date: Dec. 22, 2011

(30) Foreign Application Priority Data
Mar. 31, 2009 (IN) 681/DEL/2009

(51) Int. Cl. A61K 9/127; A61P 43/00; A61K 31/7105; C12N 5/10; B82Y 5/00

(52) U.S. Cl. 424/450; 514/44 A; 435/375; 977/773

(57) ABSTRACT

The present invention provides method and pharmaceutical composition for efficient delivery of siRNA (small interfering ribonucleic acids) into cultured mammalian cells. In addition, the present invention provides methods and compositions for knocking down the expression of a specific target gene by treating cells with the formulations comprising cationic amphiphile, a neutral colipid and a small RNA molecule. We demonstrate that our method delivers siRNA efficaciously into animal cells for the purpose of RNA interference. The area of medical science that is likely to benefit most from the present invention is RNAi therapeutics.

### FIELD OF INVENTION

The present invention provides amphoteric liposomal compositions for cellular delivery of small RNA molecules for use in RNA interference. The present invention also provides the use of amphoteric pharmaceutical composition for silencing expression of genes through RNA-interference (RNAi). The area of medical science that is likely to benefit most from the present invention is therapy of inherited diseases through RNA interference.

### BACKGROUND AND PRIOR ART INFORMATION

RNAi therapeutics are emerging new ways to combat human diseases through silencing of undesired gene expressions. The discovery of long double-stranded RNA mediated RNAi in the worm (Fire, A. et al. Nature 1998; 391:806-811) followed by demonstration of RNAi mediated by small interfering RNA (siRNA) in mammalian cells (Elbashir, S. M. et al. Nature 2001; 411:494-498) have generated an unprecedented global interest in RNAi therapeutics. The small RNA molecules involved in RNAi pathways include small interfering RNAs (siRNAs) and microRNAs (miRNAs) with the latter deriving from imperfectly paired non-coding hairpin RNA structures those are naturally transcribed by the genome (Meister, G. and Tuschi, T. Nature 2004; 431:343-349; Kim, D. H. and Rossi, J. J. Nature Rev Genet 2007; 8:175-184). siRNA mediates gene silencing through sequence specific cleavage of perfectly complementary messenger RNA (mRNA) whereas gene silencing by miRNAs are mediated through translational repression and transcript degradation for imperfectly complementary target messenger RNAs. The steps involved in the endogenous production of microRNAs include: (a) processing of RNAs with stems or short-hairpin structures (encoded in the intragenic regions or within the introns) in the nucleus to form precursor RNA molecules called pre-microRNAs; (b) export of the pre-microRNAs from the nucleus into the cell cytoplasm; (C) further shortening and processing of the pre-miRNAs by an RNase III enzyme called Dicer to produce an imperfectly matched, double-stranded miRNA (Kim, D. H. and Rossi, J. J. Nature Rev Genet 2007; 8:175-184; He, L. and Hannon, G. J. Nature Rev Genet 2004; 5:522-531). Dicer similarly processes long, perfectly matched dsRNA into siRNAs. A multi-enzyme complex including the Argonoute 2 (AGO2) and the RNA-induced silencing complex (RISC) binds to either the microRNA duplex or the siRNA duplex and discards one strand forming an activated complex containing the guide or antisense strand (Mantranga, C. et al. Cell 2005; 123:607-620). The activated AGO2-RISC complex then induces silencing of gene expression by binding with the mRNA strand of complementary sequence followed by its subsequent cleavage. Gene silencing through mRNA cleavage owes its potency to the rapid nucleolytic degradation of the mRNA fragments. Once the mRNA is degraded, the activated RISC complex becomes free to bind and cleave another target mRNA in a catalytic fashion (Hutvagner, C and Zamore, P. D. Science 2002; 297:2056-2060).

The first in vivo study on RNAi-based therapeutics was disclosed in an animal disease model in 2003 (Song, E. et al. Nat. Med. 2003; 9:347-351). Ever since then, a plethora of in vivo studies on RNAi therapeutics have been reported. siRNA mediated inhibitions of vascular endothelial growth factor have been demonstrated to be capable of suppressing tumor vascularization and growth in mice (Filleur, S. et al. Cancer Res. 2003; 63:3919-3922, Takei, Y. et al. Cancer Res. 2004; 64:3365-3370) as well as in inhibiting ocular neovascularization in a mouse model (Reich, S J et al. Mol. Vis. 2003; 9:210-216). Galun, E. demonstrated that replication of hepatitis B virus in mice can be inhibited by siRNA (Mol. Ther. 2003; 8:769-776). Small interfering RNA directed against beta-catenin has been shown to inhibit the in vitro and in vivo growth of colon cancer cells (Verma, U N et al. Clin. Cancer Res.2003; 9:1291-1300). Caspase 8, small interfering RNA has been shown to be capable of preventing acute liver failure in mice (Zender, L. et al. Proc. Natl. Acad. Sci. USA. 2003; 100:7797-7802). Inhibition of influenza virus production in virus-infected mice has been achieved through RNA interference (Ge, Q. et al. Proc. Natl. Acad. Sci. USA. 2004; 101:8676-8681, Tompkins, S M et al. Proc. Natl. Acad. Sci. USA. 2004; 101:8682-8686). Use of siRNA targeting Fas has been used to protect mice against renal ischemia-reperfusion injury (Hamar, P. et al. Proc. Natl. Acad. Sci. USA. 2004; 101:14883-14888). Small interfering RNA, upon nasal administration, has been shown to inhibit respiratory viruses (Bitko, V. et al. Nat. Med. 2005; 11:50-55). siRNA targeting Raf-1 can inhibit tumor growth both in vitro and in vivo (Leng, Q. and Mixson, A J. Cancer Gen. Ther. 2005; 12:682-690). Small interfering RNA against CXCR-4 blocks breast cancer metastasis (Liang Z. et al. Cancer Res. 2005; 65:967-971). Intravesical administration of siRNA targeting PLK-1 successfully prevented the growth of bladder cancer (Nogawa, M. et al. J. Clin. Invest. 2005; 115:978-985). Suppression of ocular neovascularization with siRNA targeting VEGF receptor 1 has been achieved (Shen, J. et al. Gene Ther. 2006; 13:225-234). Selective gene silencing in activated leukocytes has been demonstrated by targeting siRNA to the integrin lymphocyte function-associated antigen (Peer, D. et al. Proc. Natl. Acad. Sci. USA. 2007; 104:4095-4100).

Beyond identifying an active target sequence, a key challenge in the field of RNAi therapeutics is ensuring efficient delivery of small interfering RNAs inside the cell cytoplasm. Efficient intracellular delivery of biologically active compounds have previously been accomplished using liposomes, microscopic fatty bubbles of amphiphilic molecules which contain both hydrophobic (water hating) and hydrophilic (water loving) regions in their molecular architectures. Several methods for complexing biologically active compounds with liposomes have been developed. For instance, DOTMA (N-1-(2,3-dioleyloxy)propyl]-N,N,N-trimethylammonium chloride) was the first cationic amphiphile used to deliver biologically active polynucleotides (Feigner et al. Proc. Natl. Acad. Sci. USA. 1987; 84:7413-7417). Ever since then, a plethora of cationic amphiphiles have been used in delivering polynucleotides into the cell cytoplasm (Karmali, P. P. and Chaudhuri, A. Med. Res. Rev. 2007; 27:696-722 and the references cited therein). Cationic liposomes in particular, are least immunogenic. Manufacturing a greater degree of control can be exercised over the lipid's structure on a molecular level and the products can be highly purified. Use of cationic liposomes does not require any special expertise in handling and preparation techniques. Cationic liposomes can be covalently grafted with receptor specific ligands for accomplishing targeted gene delivery. Such multitude of distinguished favorable clinical features are increasingly making cationic liposomes as the non-viral transfection vectors of choice for delivering polynucleotide into body cells.

The following references are examples of cationic liposomes and their formulations that are known in the art to be useful for enhancing the intracellular delivery of genetic materials.

U.S. Pat. Nos. 4,897,355 and 4,946,787 (1990) reported the synthesis and use of N-[.omega..(.omega.-1)-dialkyloxy]-and N-[..omega..(.omega.-1)-dialkenyloxy]-alk-1-yl-N,N,N-tetrasubstituted ammonium amphiphiles and their pharmaceutical formulations as efficient transfection vectors.

Leventis, R. and Silvius, J. R Biochim. Biophys. Acta. 1990; 1023: 124-132 reported the interactions of mammalian cells with lipid dispersions containing novel metabolizable cationic amphiphiles.

U.S. Pat. No. 5,264,618 (1993) reported the synthesis and use of additional series of highly efficient cationic lipids for intracellular delivery of biologically active molecules.

Felgner et al. J. Biol. Chem. 1994; 269: 2550-2561 reported enhanced gene delivery and mechanistic studies with a novel series of cationic lipid formulations.

U.S. Pat. No. 5,283,185 (1994) reported the synthesis and use of 3β[N—(N¹,N¹-dimethylaminoethane)carbamoyl]cholesterol, termed as “DC-Chol” for delivery of a plasmid carrying a gene for chloramphenicol acetyl transferase into cultured mammalian cells.

U.S. Pat. No. 5,283,185 (1994) reported the use of N-[2-[[2,5-bis[(3-aminopropyl)amino]-1-Oxopentyl]aminoethyl]-N,N-dimethyl-2,3-bis-(9-octadecenyloxy)-1-Propanaminium tetra(trifluoroacetate), one of the most widely used cationic lipids in gene delivery. The pharmaceutical formulation containing this cationic lipid is sold commercially under the trade name “Lipofectamine”.

Solodin et al. Biochemistry 1995; 34: 13537-13544 reported a novel series of amphilic imidazolinium compounds for in vitro and in vivo gene delivery.

Wheeler et al. Proc. Natl. Acad. Sci. U.S.A. 1996; 93: 11454-11459 reported a novel cationic lipid that greatly enhances plasmid DNA delivery and expression in mouse lung.

U.S. Pat No. 5,527,928 (1996) reported the synthesis and the use of N,N,N,N-tetramethyl-N,N-bis(hydroxy ethyl)-2,3-di(oleolyoxy)-1,4-butanediammonim iodide i.e pharmaceutical formulation as transfection vector.

U.S. Pat. No. 5.698,721 (1997) reported the synthesis and use of alkyl O-phosphate esters of diacylphosphate compounds such as phosphatidylcholine or posphatidylethanolamine for intracellular delivery of macromolecules.

U.S. Pat. Nos. 5,661,018; 5,686,620and 5,688,958 (1997) disclosed a novel class of cationic phospholipids containing phosphotriester derivatives of phosphoglycerides and sphingolipids efficient in the lipofection of nucleic acids.

U.S. Pat. No. 5,614,503 (1997) reported the synthesis and use of an amphiphatic transporter for delivery of nucleic acid into cells, comprising an essentially nontoxic, biodegradable cationic compound having a cationic polyamine head group capable of binding a nucleic acid and a cholesterol lipid tail capable of associating with a cellular membrane.

U.S. Pat. No. 5,705,693 (1998) disclosed the method of preparation and use of new cationic lipids and intermediates in their synthesis that are useful for transfecting nucleic acids or peptides into prokaryotic or eukaryotic cells. These lipids comprise one or two substituted arginine, lysine or ornithine residues, or derivatives thereof, linked to a lipophilic moiety.

U.S. Pat. No.5,719,131 (1998) has reported the synthesis of a series of novel cationic amphiphiles that facilitate transport of genes into cells. The amphiphiles contain lipophilic groups derived from steroids, from mono or dialkylamines, alkylamines or polyalkylamines.

U.S. Pat. No. 5,527,928, (1996) reported on the synthesis and transfection biology of a novel cationic lipid namely, N,N,N′,N′-tetramethyl-N,N′-bis (2-hydroxyethyl)-2,3-di(oleoyloxy)-1,4-butaneammonium iodide.

U.S. Pat. No. 6,541,649 (2003) disclosed novel cationic amphiphiles containing N-hydroxyalkyl head-group and its formulation for intracellular delivery of genetic materials.

U.S. Pat. No. 6, 503, 945 (2003) disclosed novel cationic amphiphiles containing N-hydroxyalkyl head-group and its formulation for intracellular delivery of genetic materials.

U.S. Pat. No. 7,101,995 (2006) disclosed a composition with low toxicity comprising an amphipathic compound, a polycation and a siRNA. The composition can be used for delivering siRNA into the cytoplasm of cultured mammalian cells.

U.S. Pat. No. 7,157,439 (2007) disclosed methods and compositions for improving and/or controlling wound healing by applying a wound care device comprising HoxD3 and HoxA3 and/or HoxB3 novel cationic amphiphiles containing N-hydroxyalkyl head-group and its formulation for intracellular delivery of genetic materials.

### OTHER PUBLICATIONS

Behr, J. P. et al. Proc. Natl. Acad. Sci. USA, 1989; 86: 124-132.

Levantis, R. et al. Biochim. Biophys. Res. Commun. 1991; 179: 280-285.

Akao, T. et al. Biochem. Mol. Biol. Int. 1994; 34: 915-920.

Feigner, J. H. et al. Proc. Natl. Acad. Sci. USA. 1996; 93: 11454-11459.

Bennett, M. J. et al. J. Med. Chem. 1997; 40: 4069-4078.

Blessing, T. et al. J. Am. Chem. Soc. 1998; 120: 8519-8520.

Wang, J. et al. J. Med. Chem. 1998; 41: 2207-2215.

Lim, Y. et al. J. Am. Chem. Soc. 1999; 121: 5633-5639.

Lim, Y. et al. J. J. Am. Chem. Soc. 2000; 122: 6524-6525.

Zhu, J. et al. J. Am. Chem. Soc. 2000; 122: 3252-3253.

Lynn, D. M.; Langer, R. J. Am. Chem. Soc. 2000; 122: 10761-10768.

Ferrari, M. E.; Rusalov, D.; Enas, J.; Wheeler, C. J.; Nuc. Acid. Res. 2001, 29, 1539-1548.

Banerjee, R.; Das, P. K.; Srilakshmi, G. V.; Chaudhuri, A.; Rao, N. M. J. Med. Chem. 1999, 42, 4292-4299.

Banerjee, R.; Mahidhar, Y. V.; Chaudhuri, A.; Gopal, V.; Rao, N. M. J. Med. Chem. 2001, 44, 4176-4185.

Singh, S. R.; Mukherjee, K.; Banerjee, R.; Chaudhuri, A.; Hait, S. K.; Moulik, S. P.; Ramadas, Y.; Vijayalakshmi, A.; Rao, N. M. Chem. Eur. J. (in press).

Floch, V.; Bolc'h, G. Le.; Gable-Guillaume, C.; Bris, N. Le.; Yaouanc, J-J.; Abbayes, H. Des.; Fe'rec, C.; Cle'ment, J-C. Eur. J. Med. Chem., 1998, 33, 923-934.

Solodin, I.; Brown, C.; Bruno, M.; Chow, C.; Jang, E-H.; Debs, R.; Heath, T. Biochemistry, 1995, 34, 13537-13544.

Mukherjee, K.; Bhattacharyya, J.; Ramakrishna, S.; Chaudhuri, A. J. Med. Chem. 2008, 51, 1967-1971.

Rajesh, M.; Sen, J.; Srujan, M.; Mukherjee, K.; Bojja, S.; Chaudhuri, A. J. Am. Chem. Soc. 2007, 129, 11408-11420.

Karmali, P. P.; Majeti, B. K.; Bojja S.; and Chaudhuri, A. Bioconjugate Chemistry 2006, 17, 159-171.

Bharat M. K.; Karmali, P. P.; Reddy, B. S.; Chaudhuri, A. J. Med. Chem. 2005, 48, 3784-3795.

Sen, J. and Chaudhuri, A. J. Med. Chem. 2005, 48, 812-820.

Sen, J. and Chaudhuri, A. Bioconjugate Chemistry 2005, 16, 903-912.

Bharat M. K.; Karmali, P. P.; Chaudhuri, A. Bioconjugate Chemistry 2005, 16, 676-684.

Mukherjee, K. M., Sen, J. and Chaudhuri, A. FEBS Letters 2005, 579, 1291-1300.

Mahidhar, Y. V., Rajesh, M.; Madhavendra, S. S.; Chaudhuri, A. J. Med. Chem. 2004, 47, 5721-5728.

Mahidhar, Y. V., Rajesh, M.; Chaudhuri, A. J. Med. Chem. 2004, 47, 3938-3948.

Valluripalli, V. K. and Chaudhuri, A. FEBS Letters 2004, 571, 205-211.

Singh, R. S.; Goncalves, C.; Sandrin, P.; Pichon, C.; Midoux, P.; Chaudhuri, A. Chemistry and Biology 2004, 11, 713-723.

Majeti, B. K.; Singh, R. S.; Yadav, S. K.; Reddy, S. B.; Ramkrishna, S.; Diwan, P. V.; Madhavendra, S. S.; Chaudhuri, A. Chemistry and Biology 2004, 11, 427-437.

Karmali, P. P.; Valluripalli, V. K.; Chaudhuri, A. J. Med. Chem. 2004, 47, 2123-2132.

Singh, R. S. and Chaudhuri, A. FEBS Letters 2004, 556, 86-90.

Kumar, V. V.; Pichon, C.; Refregiers, M.; Guerin, B.; Midoux, P.; Chaudhuri, A. Gene Therapy 2003, 10, 1206-1215.

Valluripalli, V. K.; Singh, R. S ; Chaudhuri, A. Curr: Med. Chem. 2003, 10, 1297-1306.

### OBJECTIVES OF INVENTION

The objective of the present invention is to provide amphoteric liposomal composition for improved delivery of small interfering RNA (siRNA) for use in RNA interference.

Another objective of the invention is to provide the process for delivering small RNA molecules inside the animal cells. Such delivery process comprises the preparation of a ternary complex of cationic amphiphile, neutral colipid and the small RNA molecules, associating the ternary complexes with the cells and delivering the small RNA molecules into the interior of cells.

One another objective of the present invention is to provide the use of amphoteric pharmaceutical composition for knocking down the expression of a specific target gene by treating cells with the formulations comprising cationic amphiphile, a neutral colipid and a small RNA molecule.

### SUMMARY OF THE INVENTION

The present invention provides amphoteric liposomal composition comprising cationic amphiphile, neutral colipid to deliver siRNA in mammalian cultured cells for knock down expression of target gene for the purpose of RNA interference

Accordingly, the present invention provides amphoteric liposomal composition for cellular delivery of small RNA molecules for use in RNA interference wherein the said composition comprises a cationic amphiphile having aliphatic hydrocarbon tail represented by, general formula 1 and a neutral colipid, wherein R₁═R₂=n-C₁₄H₂₉ or n-C₁₆H₃₃, R₃═—CH₃ or CH₂CH₂OH and
R₄=Guanidinyl or OH;

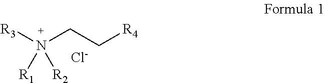

and wherein the ratio of said cationic amphiphile and neutral colipid ranges between 1:1 to 3:1.

In an embodiment of the present invention, the amphoteric liposomal composition exhibits the following characteristics:
a) stable in the pH range 2-10 for efficient delivery of siRNA
b) average size of the amphoteric liposome falling within the range of 30-250 nm
c) capable of knocking down the expression of target gene in cultured mammalian cells.

In another embodiment of the present invention, the cationic amphiphile used is selected from the group consisting of N,N-di-n-tetradecyl-N-(2-guanidinyl)ethyl-N-methylammonium chloride, N,N-di-n-hexadecyl-N-(2-guanidinyl)ethyl-N-methylammonium chloride and N,N-di-n-tetradecyl,N,N-di-(2-hydroxyethyl)ammonium chloride.

In yet another embodiment of the present invention, the cationic amphiphile used is preferably N,N-di-n-tetradecyl-N-(2-guanidinyl)ethyl-N-methylammonium chloride.

In still another embodiment of the present invention, the said cationic amphiphile preparation comprises the steps of:
a) reacting thiourea with t-butyloxycarbonyl(Boc)-anhydride in a molar ratio of 1:2 in presence of sodium anhydride in anhydrous tetrahydrofuran at temperature of 0-2 degrees C. under stirring to obtain compound, bis-N-Boc-thiourea (II);
b) reacting N-2-aminoethyl-N,N-di-n-tetradecylamine (I) with bis-N-Boc-thiourea (II) of step (a) in a molar ratio of 1:1 in presence of mercuric chloride and triethylamine (TEA) in dimethylformamide (DMF) and dichloromethane (DCM) under inert atmosphere at a temperature of 0-2 degrees C. for 40 minutes followed by purification using methanol-dichloromethane as eluent to obtain intermediate compound, N,N-di-n-tetradecyl-N-[2-(N′,N′-di-tertbutoxycarbonyl-guanidinyl]ethylamine (III);
c) reacting compound N,N-di-n-tetradecyl-N-[2-(N′,N′-di-tertbutoxycarbonylguanidinyl]ethyl amine (III) of step (b) with methyl iodide (MeI) in dichloromethane/methanol (2:1) at room temperature for overnight followed by purification using methanol-dichloromethane as eluent to obtain an intermediate compound, N,N-di-n-tetradecyl-N-[2-N′,N′-di-tertbutoxycarbonylguanidinyl]ethyl-N-methylammonium iodide, which is subjected to t-butyloxycarbonyl (Boc) deprotection using trifluoroacetic acid (TFA) in DCM and finally followed by chloride ion exchange chromatography over amberlyst A-26chloride ion exchange resin to obtain the cationic amphiphile, N,N-di-n-tetradecyl-N-[2-guanidinyl]ethyl-N-methylammonium chloride.

In a further embodiment of the present invention, the neutral colipid used is selected from the group consisting of cholesterol, fatty alcohol, phosphatidyl ethanolamine, phosphatidylcholine and sphingolipid or diacyl glycerol.

In another embodiment of the present invention, the preferred neutral colipid used is cholesterol.

In yet another embodiment of the present invention, the molar ratio of the cationic amphiphile to neutral colipid used is in the range of 1:1 to 3:1.

In still another embodiment of the present invention, the preferred molar ratio of the cationic amphiphile to neutral colipid is 1:1.

In yet another embodiment of the present invention, the amphoteric pharmaceutical composition comprises an amphoteric liposomal composition along with a nucleotide.

In another embodiment of the present invention, the nucleotide used is selected from the group of small interfering RNA (siRNA), microRNA, antisense oligonucleotide or a decoy nucleotide.

In yet another embodiment of the present invention, the preferred nucleotide used is siRNA.

In still another embodiment of the present invention, the molar ratio of cationic amphiphile to siRNA lies within the range of 1:1 to 100:1.

In another embodiment of the present invention, the preferred mole ratio of cationic amphiphile to siRNA is 50:1.

In yet another embodiment of the present invention, the amphoteric pharmaceutical composition is useful for the delivery of siRNA in cultured mammalian cells, selected from the group consisting of COS-1(African green monkey kidney cells), CHO (Chinese hamster ovary cells), HepG2 (human hepatocyte cells), RAW264.7 (mouse peritoneal macrophage cells).

In still another embodiment of the present invention, the composition comprising cationic amphiphile, neutral colipid and siRNA is useful for knocking down the expression of target gene inside cultured mammalian cells.

In a further embodiment of the present invention is provided a novel use of an amphoteric pharmaceutical composition, for knocking down the expression of target gene inside cultured mammalian cells comprising the following steps:
a) seeding cells at 1×10⁴ cells/well in 96 well plate with 100 μl of growth medium containing FBS medium followed by incubation for 24 hrs
b) forming complex of luciferase GL2 siRNA, liposome and pCMV-GL2 luciferase plasmid by
i) diluting 5-50 pmol luciferase GL2 siRNA duplex in 25 μl Opti-MEM® I Medium without serum followed by mixing and
   ii) adding diluted siRNA complex to the diluted liposome followed by gently mixing pCMV-GL@ Luciferase plasmid to siRNA-liposomal conjugate and incubating for 10-20 minutes at room temperature
   iii) adding siRNA duplex-liposome-plasmid DNA complex to each well
   iv) changing medium after 4 hrs and incubating for 30 hrs at 37 degrees in CO₂ incubator and performing assay in triplicate for knock down expression of luciferase.

### BRIEF DESCRIPTION OF DRAWINGS

FIG. 1 is a schematic representation of the synthetic scheme followed in preparing the cationic amphiphile N,N-di-n-tetradecyl-N-(2-guanidinyl)ethyl-N-methylammonium chloride containing the guanidinium head-groups.

FIG. 2. Inverted fluorescence micrographs of the COS-1 cells transfected with complex of fluorescein labeled siRNA, cationic liposomes prepared with equimolar amounts of N,N-di-n-tetradecyl-N-(2-guanidinyl)ethyl-N-methylammonium chloride (cationic amphiphile) and cholesterol. Cationic amphiphile:siRNA mole ratios were maintained at 50:1. A-C: Images for cells transfected with cationic liposomes prepared with equimolar amounts of N,N-di-n-tetradecyl-N-(2-guanidinyl)ethyl-N-methylammonium chloride and cholesterol (A. phase contrast bright field image; B. Fluorescence micrograph and C. overlay images). D-F: images for cells transfected with commercially available Lipofectamine 2000 (D. phase contrast bright field image; E. fluorescence micrograph and F. overlay images). (Magnification: 60×).

FIG. 3. Inverted fluorescence micrographs of the RAW264.7 cells transfected with complex of fluorescein labeled siRNA, cationic liposomes prepared with equimolar amounts of N,N-di-n-tetradecyl-N-(2-guanidinyl)ethyl-N-methylammonium chloride (cationic amphiphile) and cholesterol. Cationic amphiphile:siRNA mole ratios were maintained at 50:1. A-C: images for cells transfected with cationic liposomes prepared with equimolar amounts of N,N-di-n-tetradecyl-N-(2-guanidinyl)ethyl-N-methylammonium chloride and cholesterol (A. phase contrast bright field image; B. Fluorescence micrograph and C. overlay images). D-F: images for cells transfected with commercially available Lipofectamine 2000 (D. phase contrast bright field image; E. fluorescence micrograph and F. overlay images). (Magnification: 60×).

FIG. 4. Inverted fluorescence micrographs of the CHO cells transfected with complex of fluorescein labeled siRNA, cationic liposomes prepared with equimolar amounts of N,N-di-n-tetradecyl-N-(2-guanidinyl)ethyl-N-methylammonium chloride and cholesterol. Cationic amphiphile:siRNA mole ratios were maintained at 50:1. A-C: images for cells transfected with cationic liposomes prepared with equimolar amounts of N,N-di-n-tetradecyl-N-(2-guanidinyl)ethyl-N-methylammonium chloride and cholesterol (A. phase contrast bright field image; B. Fluorescence micrograph and C. overlay images). D-F: images for cells transfected with commercially available Lipofectamine 2000 (D. phase contrast bright field image; E. fluorescence micrograph and F. overlay images). (Magnification: 60×).

FIG. 5. Inverted fluorescence micrographs of the HepG2 cells transfected with complex of fluorescein labeled siRNA, cationic liposomes prepared with equimolar amounts of N,N-di-n-hexadecyl-N-(2-guanidinyl)ethyl-N-methylammonium chloride and cholesterol. Cationic amphiphile: siRNA mole ratios were maintained at 50:1. A-C: images for cells transfected with cationic liposomes prepared with equimolar amounts of N,N-di-n-tetradecyl-N-(2-guanidinyl)ethyl-N-methylammonium chloride and cholesterol (A. phase contrast bright field image; B. Fluorescence micrograph and C. overlay images). D-F: images for cells transfected with commercially available Lipofectamine 2000 (D. phase contrast bright field image; E. fluorescence micrograph and F. overlay images). (Magnification: 60×).

FIG. 6. Representative efficiencies of the presently described formulation comprising of N,N-di-n-tetradecyl-N-(2-guanidinyl)ethyl-N-methylammonium chloride, cholesterol, luciferase GL2 siRNA in knocking down the expression of the firefly luciferase GL2 gene in CHO cells. Cationic amphiphile: siRNA mole ratios were maintained at 50:1.

### DETAILED DESCRIPTION OF THE INVENTION

The present invention provides amphoteric liposomal composition for delivering small RNA molecules inside the cytoplasm of cultured mammalian cells with high efficiency and low toxicity. In addition, the present invention also provides amphoteric pharmaceutical composition for knocking down the expression of a specific target gene by treating cells with the composition comprising cationic amphiphile, a neutral colipid and small interfering RNA molecule. We demonstrate that our method delivers siRNA efficaciously into animal cells for the purpose of RNA interference.

The following examples are given by way of illustration of the present invention and therefore should not be construed to limit the scope of the present invention.

### EXAMPLE 1

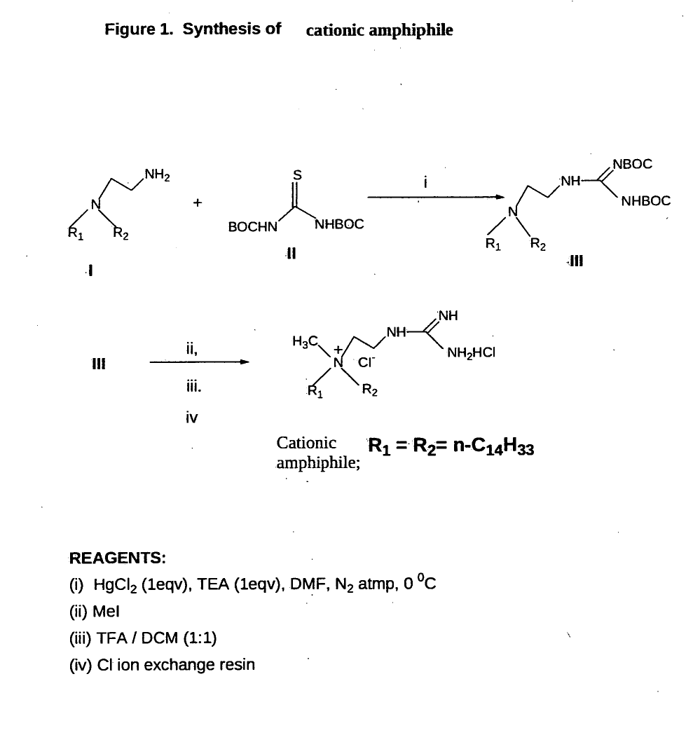

Synthesis of the cationic amphiphile (FIG. 1). Cationic amphiphile was synthesized following the procedures depicted schematically in FIG. 1.

Synthesis of Cationic Amphiphile

Step-i. Synthesis of N,N-di-n-tetradecyl-N-[2-(N′,N′-di-tertbutoxycarbonyl-guanidinyl]ethyl amine (III, FIG. 1). Mercuric chloride (0.28 g, 1.0 mmol) was added to a mixture of N-2-aminoethyl-N,N-di-n-tetradecylamine (I, 0.49 g, 1.1 mmol), bis-N-Boc-thiourea (II, 0.08 g, 1.1 mmol, prepared conventionally by reacting one equivalent of thiourea with 2 equivalents of Boc-anhydride in presence of 2 equivalents of sodium hydride in anhydrous tetrahydrofuran at temperature of 0-2 degrees C. under stirring) and triethylamine (0.21 g, 2.1 mmol) dissolved in dry DMF (5 ml) and dry DCM (2 ml). The resulting mixture was stirred at 0° C. under nitrogen atmosphere for 40 minutes, diluted with ethyl acetate (20 ml) and filtered through a pad of celite. The filtrate was sequentially washed with water (2×20 ml) and brine solution (2×20 ml), dried over anhydrous sodium sulfate, filtered and the solvent from the filtrate removed by rotary evaporation. The residue upon column chromatographic purification with 60-120 mesh silica gel using 2-2.5% methanol-dichloromethane (v/v) as eluent afforded 0.37 g of the pure title compound III (70%, R_{f}=0.8, 10% methanol-dichloromethane, v/v).

¹H NMR (200 MHz, CDCl₃): δ/ppm=0.9 [t, 6H, CH₃—(CH₂)₁₃—]; 1.2-1.4 [bs, 44H, —(CH₂)₁₁—]; 1.4-1.6 [2s, 18H, —CO—O—C(CH₃)₃]; 2.4-2.7 [bm, 6H, —N(—CH₂—CH₂—)₂—; —N—CH₂—CH₂—NH—]; 3.4-3.6 [m, 2H, —N—CH₂—CH₂—NH—]; 8.6 [t, 1H, —CH₂—NH—]; 11.4 [s, 1H, —NHBOC].

Step-ii. Synthesis of N,N-di-n-tetradecyl-N-[2-(N′,N′-di-tertbutoxycarbonylguanidinyl]ethyl-N-methylammonium iodide (FIG. 1). The intermediate III obtained above in step i was dissolved in 3 ml dichloromethane/methanol (2:1, v/v) and 3 ml methyl iodide was added. The solution was stirred at room temperature overnight. Solvent was removed on a rotary evaporator. The residue upon column chromatographic purification with 60-120 mesh size silica gel and 3% methanol in dichloromethane (v/v) as eluent afforded 0.35 g of the title compound (80% yield, R_{f}=0.29, 10% methanol in dichloromethane, v/v).

¹H NMR (200 MHz, CDCl₃): δ/ppm=0.9 [t, 6H, CH₃—(CH₂)₁₃—]; 1.2-1.3 [m, 36H, —CH₃(CH₂)₉—]; 1.4-1.6 [2s, 18H, —CO—O—C(CH₃)₃]; 1.65 [m, 4H, —N⁺(—CH₂—CH₂—)₂]; 3.3 [s, 3H,—N⁺—CH₃]; 3.4 [m, 4H, —N⁺(—CH₂—CH₂—)₂]; 3.6 [m, 2H, —N⁺—CH₂—CH₂—NH—]; 3.8 [m, 2H, —N⁺—CH₂—CH₂—NH—]; 8.4 [t, 1H, —CH₂—NH—]; 11.3 [s, 1H,—NHBOC].

Steps-iii & iv. Synthesis of N,N-di-n-tetradecyl-N-[2-guanidinyl]ethyl-N-methylammonium chloride (cationic amphiphile, FIG. 1).

The intermediate obtained above in step ii was dissolved in dry DCM (2 ml) and TFA (2 ml) was added to the solution at 0° C. The resulting solution was left stirred at room temperature overnight to ensure complete deprotection. Excess TFA was removed by flushing nitrogen to give the title compound as a trifluoroacetate salt. Column chromatographic purification using 60-120 mesh size silica gel and 12-14% (v/v) methanol-chloroform as eluent followed by chloride ion exchange chromatography over amberlyst A-26 chloride ion exchange resin afforded 0.16 g of the pure lipid A (90% yield, R_{f}=0.3, 10% methanol in chloroform, v/v).

¹H NMR (200 MHz, CDCl₃): δ/ppm=0.9 [t, 6H, CH₃—(CH₂)₁₄—]; 1.2-1.3 [m, 44H, —CH₃(CH₂)₁₁—]; 1.5-1.7 [m, 4H, —N⁺(—CH₂—CH₂—)₂]; 3.0 [s, 3H,—N⁺—CH₃]; 3.1 [m, 4H, —N⁺(—CH₂—CH₂—)₂]; 3.5 [m, 2H, —N⁺—CH₂—CH₂—NH—]; 3.7 [m, 2H, —N⁺—CH₂—CH₂—NH—]; 7.4[bs, 4H, —NH₂⁺]; 8.7 [bs, 1H, —CH₂—NH].

LSIMS (lipid A): m/z: 510 [M+1⁺] (calcd for C₃₂H₆₉N₄, 82%).

### Example 2

Evaluation of siRNA delivery efficacies of the amphoteric composition containing N,N-di-n-tetradecyl-N-[2-guanidinyl]ethyl-N-methylammonium chloride (FIG. 1) done in four cells including COS-1, RAW264.7, CHO and HepG2 cells.

Cells were seeded at a density of 40,000 cells/well in a 24-well plate for 18 hrs before transfection in 500 μl of growth medium such that the well became 30-50% confluent at the time of transfection. For each well to be transfected, siRNA duplex-Liposome complexes were prepared as follows:

a) 20 pmol fluorescently labeled siRNA duplex namely, control(non-sil) siRNA, Fluorescein (Catalog No. 1022079, QIAGEN, USA) was diluted in 50 μl Opti-MEM® I reduced serum Medium without serum in the well of the tissue culture plate and was mixed gently.

b) Liposomes were prepared by dissolving the cationic amphiphile and the neutral co-lipid, i.e., cholesterol in the appropriate mole ratio in a mixture of methanol and chloroform in a glass vial. The solvent was removed with a thin flow of moisture free nitrogen gas and the dried lipid film was then kept under high vacuum for 8 hrs. The dried lipid film was hydrated in sterile deionized (RNAse free) water in a total volume of 1 ml at Guanidinylated cationic amphiphile concentration of 1 mM for a minimum of 12 hrs. Liposomes were vortexed for 1-2 minutes to remove any adhering lipid film and sonicated in a bath sonicator (ULTRAsonik 28×) for 2-3 minutes at room temperature to produce multilamellar vesicles (MLV). MLVs were then sonicated with a Ti-probe (using a Branson 450 sonifier at 100% duty cycle and 25 W output power) for 1-2 minutes to produce small unilamellar vesicles (SUVs) as indicated by the formation of a clear translucent solution. 1 μl liposome was then added to each well containing the diluted siRNA molecules, mixed gently and was incubated for 10-20 minutes at room temperature.

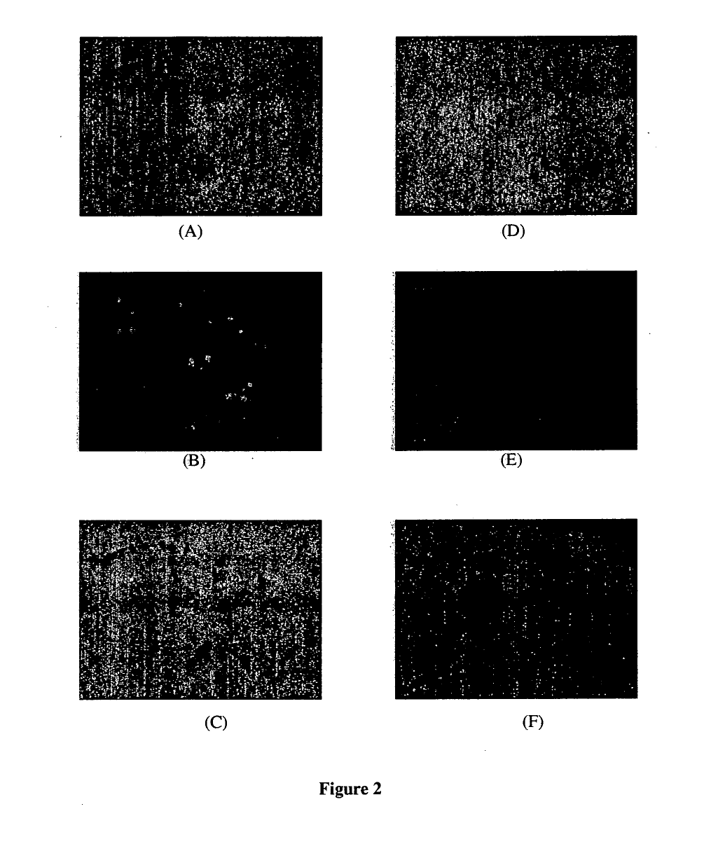

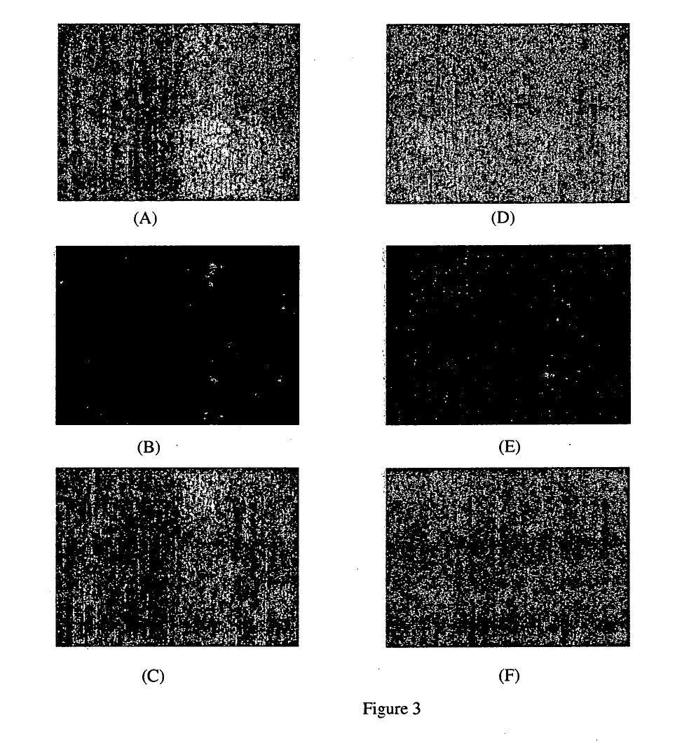

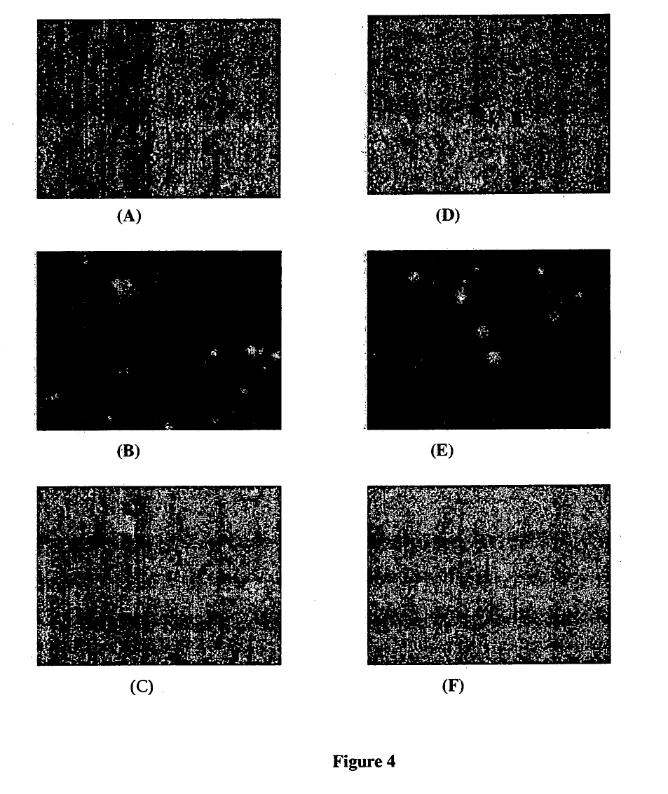

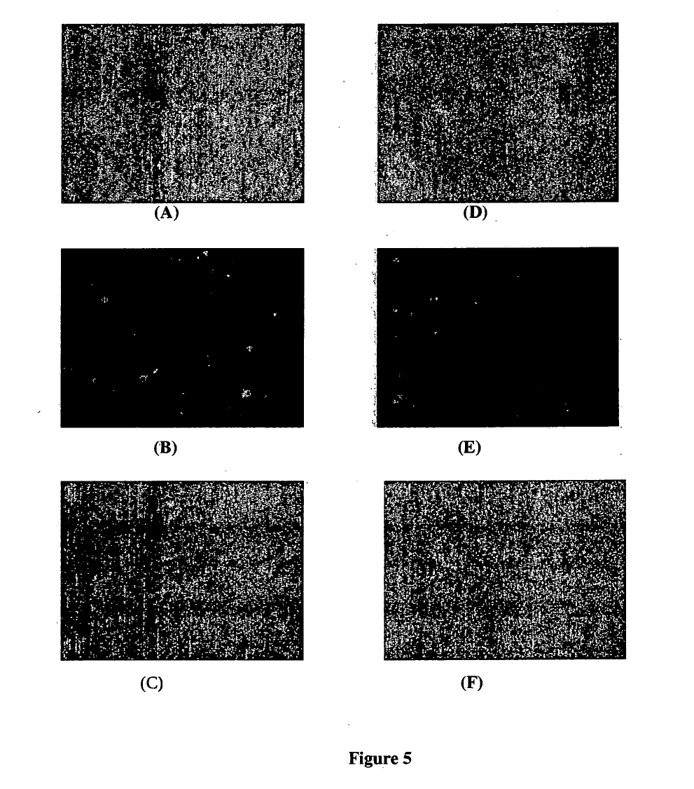

The siRNA duplex-Liposome complexes obtained above were added to each well containing 40,000 cells. After incubation of the cell plates in a humidified atmosphere containing 5% CO₂ at 37° C. for 4 hrs, 200 μl of growth medium containing 10% FBS (CM1X) were added to cells. After 8 hrs, the medium was removed completely from the wells and cells were washed with PBS (200 μl). PBS was discarded and micrographs were taken on fresh PBS (200 μl). The fluorescently labeled cells were observed under an inverted fluorescence microscope (Nikon, Japan). As depicted in FIGS. 2-5, the cellular uptake efficiencies of the fluorescently labeled siRNA:cationic amphiphile:Cholesterol ternary complexes were found to be better than or comparable to that of Lipofectamine 2000 (Invitrogen, USA), a commercially available widely used siRNA delivery reagent in four cultured cells including COS-1, RAW264.7, CHO and HepG2 cells.

### Example 3

Knocking down the expression of firefly luciferase GL2 gene in CHO cells by delivering luciferase GL2 siRNA with the help of the formulation containing equimolar amounts of N,N-di-n-tetradecyl-N-[2-guanidiny]ethyl-N-methylammonium chloride (cationic amphiphile, FIG. 1) and cholesterol.

One day before transfection, cells were seeded at 1×10⁴ cells/well in 96-well plates with 100 μl of growth medium containing 10% FBS medium and incubated for 24 hrs. Cells were 50-60% confluent before transfection. The complex of luciferase GL2 siRNA, liposome and pCMV-GL2 Luciferase plasmid (obtained as a generous gift from the laboratory of Professor Leaf Huang, University of North Carolina, Chapel Hills, USA) was prepared as follows:
a. 5-50 pmol luciferase GL2 siRNA duplex was diluted in 25 μl Opti-MEM® I Reduced

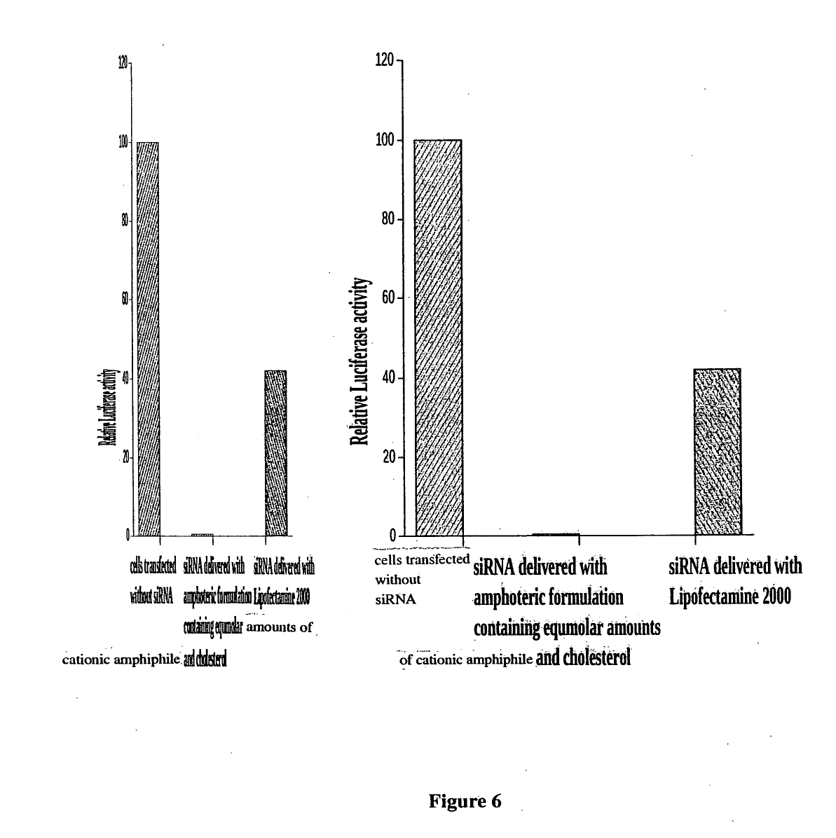

Serum
Medium without serum and was mixed gently.
b. Liposomes prepared using equimolar cationic amphiphile and cholesterol was mixed gently before use. 1.38 μl of liposome (containing 1 mM cationic amphiphile) was then diluted with 115 μl of Opti-MEM® I Reduced Serum Medium and mixed gently.
c. Diluted siRNA duplex was added to the diluted liposome and mixed gently. 0.9 μg of the pCMV-GL2Luciferase plasmid (9 μl of 0.1 μg/μl stock plasmid) was added to the siRNA-liposomal conjugate and incubated for 10-20 minutes at room temperature. This gave a final volume of 150 μl siRNA duplex-liposome-plasmid DNA complex.
d. 50 μl of the siRNA duplex-liposome-plasmid DNA complex prepared above was added to each well. Medium was changed after 4 h and the cells were incubated for 30 hours at 37° C. in a CO₂ incubator and assayed for knock-down of luciferase expression. Each gene knock-down experiment with siRNA was done in triplicate using Microplate Luminometer (FLₓ800, Bio-Tek Instruments, USA). As depicted in FIG. 6, the efficacy of the presently disclosed amphoteric formulation containing equimolar amounts of cationic amphiphile and cholesterol was superior to that of commercially available Lipofectamine 2000 (Invitrogen, USA) in knocking down the expression of the firefly luciferase gene (GL2) in CHO cells.

Applications

The area of medical science that is likely to benefit most from the present invention is RNAi therapeutics. The formulations described in the present invention can be exploited for efficient delivery of small RNA molecules into the interiors of animal cells for use in RNA interference. In addition, the present invention provides method and composition for knocking down the expression of a specific target gene by treating cells with the formulations comprising cationic amphiphile, neutral colipid and small RNA molecule.

1-17. (canceled)

18. An amphoteric liposomal composition for use in RNA inference comprising:
i) a cationic amphiphile having an aliphatic hydrocarbon tail represented by formula 1, 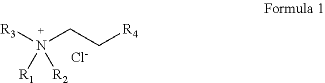
wherein R₁═R₂=n-C₁₄H₂₉ or n-C₁₆H₃₃, R₃═—CH₃ or —CH₂CH₂OH and R₄=guanidinyl or OH, and
ii) a neutral colipid,
wherein the ratio of said cationic amphiphile and neutral colipid ranges from 1:1 to 3:1.

19. The amphoteric liposomal composition as claimed in claim 18, wherein said liposomal composition:
a) is stable at a pH range of 2 to 10;
b) comprises amphoteric liposomes having an average size of from 30 to 250 nm.

20. The amphoteric liposomal composition of claim 18, wherein the cationic amphiphile is selected from the group consisting of N,-N-di-n-tetradecyl-N-(2-guanidinyl)ethyl-N-methylammonium chloride, N,N-di-n-dexadecyl-N-(2-guanidinyl)ethyl-N-methylammonium chloride and N,N-di-n-tetradecyl,N,N-di-(2-hydroxyethyl)ammonium chloride.

21. The amphoteric liposomal composition as claimed in claim 20, wherein the cationic amphiphile used is N,N-di-n-tetradecyl-N-(2-guanidinyl)ethyl-N-methylammonium chloride.

22. Process for preparing the amphoteric liposomal composition of claim 18, comprising of:
a) reacting thiourea with t-butyloxycarbonyl(Boc)-anhydride at a molar ratio of 1:2 in the presence of sodium anhydride in anhydrous tetrahydrofuran at a temperature of from 0 to 2° C., while stirring to obtain bis-N-Boc-Thiourea (II);
b) reacting N-2-aminioethyl-N,N-di-n-tetradecylamine (I) with bis-N-Boc-thiourea (II) of step (a) at a molar ratio of 1:1 in the presence of mercuric chloride and triethylamine (TEA) dissolved in dimethylformamide (DMF) and dichloromethane (DCM) under an inert atmosphere at a temperature of from 0-2° C. for 40 minutes with stirring followed by purification using methanol-dichloromethane as an eluant to obtain N,N-di-n-tetradecyl-N[2-(N′,N′-di-tertbutoxycarbonyl-guanidinyl]ethylamine (III);
c) reacting N,N-di-n-tetradecyl-N-[2-(N′,N′-di-tertbutocxycarbonylguanidinyl]amine (III) of step (b) with methyl iodide (MeI) in dichloromethane/methanol (2:1) at room temperature overnight followed by purification using methanol-dichloromethane as eluant to obtain N,N-di-n-tetradecyl-N-[2-N′,N′-di-tertbutoxycarbonylguanidinyl]ethyl-N-methylammonium iodide, followed t-butyloxycarbonyl (Boc) deprotection using trifluoroacetic acid (TFA) in DCM and chloride ion exchange chromatography over amberlyst A-26 chloride ion exchange resin to obtain N,N-di-n-tetradecyl-N-[2-guanidinyl]ethyl-N-methylammonium chloride.

23. The amphoteric liposomal composition of claim 18, wherein the neutral colipid is selected from the group consisting of cholesterol, a fatty alcohol, phosphatidyl ethanolamine, phosphatidylcholine and sphingolipid and a diacyl glycerol.

24. The amphoteric liposomal composition of claim 23, wherein said neutral colipid is cholesterol.

25. The amphoteric liposomal composition of claim 18, further comprising a nucleotide.

26. The amphoteric liposomal composition of claim 25, wherein the nucleotide is selected from the group of small interfacing RNA (siRNA), microRNA, a antisense oligonucleotide and a decoy nucleotide.

27. The amphoteric liposomal composition of claim 26, wherein the nucleotide is siRNA.

28. The amphoteric liposomal composition of claim 27, wherein the molar ratio of cationic amphiphile to siRNA is from 1:1 to 100:1.

29. The amphoteric liposomal composition of claim 28, wherein the molar ratio of cationic amphiphile to siRNA is 50:1.

30. A method for knocking down expression of target gene a cultured mammalian cell comprising:
a) seeding mammalian cells at 1×10⁴ cells/well in 96 well plate with 100 μl of growth medium containing RBS medium followed by incubation for 24 hrs,
b) forming a complex of luciferase GL2 siRNA the amphoteric liposomal composition of claim 1 pCMV-GL2 luciferase plasmid by:
i) diluting 5-50 pmol luciferase GL2 siRNA duplex in 25 μl Opti-MEM® I Medium without serum followed by mixing,
ii) adding diluted siRNA complex to the diluted liposome followed by gently mixing pCMV-GL@ Luciferase plasmid to siRNA-liposomal conjugates and incubating for 10-20 minutes at room temperature,
iii) adding siRNA duplex-liposome-plasmid DNA complex to each well,
iv) changing medium after 4 hrs and incubating for 30 hrs at 37 degrees in CO₂ incubator and performing assay in triplicate for knock down expression of luciferase.
